(19) United States
(12) Patent Application Publication
(10) Pub. No.: US 2024/0374673 A1
(43) Pub. Date: Nov. 14, 2024

(54) PHARMACEUTICAL COMPOSITION FOR PREVENTING OR TREATING INFLAMMATORY SKIN DISEASE

(71) Applicant: 3BIGS CO., LTD., Busan (KR)

(72) Inventors: Junhyung Park, Hwaseong-si, Gyeonggi-do (KR); Hoyong Chung, Incheon (KR); Sathishkumar Natarajan

(73) Assignee: 3BIGS CO., LTD., Busan (KR)

(21) Appl. No.: 18/569,278

(22) PCT Filed: Mar. 29, 2022
(86) PCT No.: PCT/KR2022/004434

§ 371 Date: Dec. 12, 2023

(30) Foreign Application Priority Data
Jun. 16, 2021 (KR) 10-2021-0078174

(51) Int. Cl. A61K 38/07; A61K 8/64; A61Q 19/00

(52) U.S. Cl. CPC A61K 38/07; A61K 8/64; A61Q 19/00

(57) ABSTRACT

The present invention relates to pharmaceutical composition for preventing or treating inflammatory skin diseases, containing a peptide exhibiting anti-inflammatory activity as an active ingredient. As it is confirmed that the synthesized novel peptide inhibits the secretion or expression of inflammation-inducing cytokines in an experimental group of human epidermal cells treated with TNF-α/IFN-γ which induces inflammation, inhibits the phosphorylation of major proteins of a JAK/STAT signaling pathway among the signaling pathways induced by TNF-α/IFN-γ treatment, reduces skin inflammation without weight change in an animal model in which chronic contact dermatitis is induced, and significantly reduces the size of the spleen and lymph nodes, which are organs majorly involved in the immune response, compared to a positive control group, the composition containing the peptide as an active ingredient may be provided as a therapeutic agent for contact dermatitis or a cosmetic composition for improving dermatitis.

### TECHNICAL FIELD

The present disclosure relates to a pharmaceutical composition and cosmetic composition for preventing or treating inflammatory skin diseases, including a peptide exhibiting an anti-inflammatory activity as an active ingredient.

### BACKGROUND ART

The skin, an organ that makes up the outside of a body, is an important physical barrier that maintains internal homeostasis while protecting the body from various external physical and chemical stimuli. The skin prevents a loss of moisture and electrolytes to the outside, maintains homeostasis such as thermoregulation and temperature, responds to stimuli such as touch, pressure, nociception, and warmth, and performs functions such as immune functions and synthesis of vitamin D.

While abnormalities that prevent the skin from performing its function are called skin diseases, thereamong, inflammatory skin diseases that bring inflammation on the skin are characterized by symptoms such as redness, swelling, warmth, and pain and classified by type into contact dermatitis, irritating contact dermatitis, allergic contact dermatitis, phototoxic and photoallergic contact dermatitis, contact urticaria syndrome, atopic dermatitis, seborrheic dermatitis, blister eczema, coin-shaped eczema, autosensitization dermatitis, housewife's eczema, epistasis dermatitis, and the like.

The inflammatory skin disease is involved with many cells such as T lymphocytes, Langerhans cells, eosinophils, and keratinocytes and caused by several elements such as cytokines, chemokines, and immunoglobulin molecules. Thereamong, keratinocytes are components constituting most of the epidermis of the skin, acting as a derivative and target of the immune response that occurs in the skin.

Contact dermatitis refers to any dermatitis that develops due to contact with foreign substances. It is divided into primary contact dermatitis, which is caused by irritation from the contact substance itself, and allergic contact dermatitis, which occurs only in people who have an allergic reaction to substances in contact with.

Primary contact dermatitis has many causative substances, including plants, metals, cosmetics, preservatives, pharmaceutical rubbers, and synthetic resins. The symptoms of primary contact dermatitis and allergic contact dermatitis are similar, showing eczema-like lesions mainly with erythema and edema. In addition, it may be accompanied by blisters or oozing, and in some cases, acne-like lesions, urticaria lesions, erythema multiforme, pigmentation, and granulomatous lesions may occur as well.

When it comes to treatment of such contact dermatitis, it is crucial to avoid contact with the causative substances, and if a reaction has already occurred after exposure, cold compression needs to be performed to dry the vesicular lesions and apply moisturizing creams and lotions. Oily ointments or creams are effective in chronic contact dermatitis, which is characterized by keratinization and lichenification, and facilitate treatment of the diseases by sealing after applying medications. In addition, if the lesion has spread throughout the body or topical medications are less effective, systemic antihistamines and corticosteroids (steroids) may be helpful.

However, drugs such as antihistamines and steroids lead to tolerance within a short period of time after administration to patients, so repeated administration after a certain period of time often fails to alleviate the symptoms in patients. Therefore, there is a need to develop drugs that can overcome side effects of antihistamines and steroids.

### DISCLOSURE OF THE INVENTION

### Technical Goals

Since an anti-inflammatory effect of a novel peptide which has an amino acid sequence represented by SEQ ID NO: 1 was identified, an object of the present disclosure is to provide the peptide as an agent for preventing or treating inflammatory skin diseases.

### Technical Solutions

The present disclosure provides a pharmaceutical composition for preventing or treating inflammatory skin diseases, including a peptide having an amino acid sequence represented by SEQ ID NO: 1 as an active ingredient.

In addition, the present disclosure provides a cosmetic composition for preventing or alleviating inflammatory skin diseases, including a peptide having an amino acid sequence represented by SEQ ID NO: 1 as an active ingredient.

### Advantageous Effects

According to the present disclosure, as it was determined that a synthesized novel peptide suppresses secretion or expression of inflammation-inducing cytokines in an experimental group of human epidermal cells treated with TNF-α/IFN-γ that induces inflammation, inhibits phosphorylation of key proteins in a JAK/STAT signaling pathway among signaling pathways induced by TNF-α/IFN-γ treatment, and reduces skin inflammation without a change in the body weight in an animal model in which chronic contact dermatitis is induced, such that a composition including the peptide as an active ingredient may be provided as a therapeutic agent for contact dermatitis or a cosmetic composition for alleviating dermatitis.

### BRIEF DESCRIPTION OF DRAWINGS

FIG. 1 shows results of PCR analysis conducted to identify an anti-inflammatory activity of a synthesized peptide, derived by treating a HaCaT cell line with TNF-α/IFN-γ (10 ng/ml) as a positive control group or TNF-α/IFN-γ in combination with a novel peptide (10 μg/ml) to identify transcription inhibitory effects of CCL17, CCL22, KDM6B, and GMCSF.

FIG. 2 shows a result of real-time PCR analysis conducted to identify an anti-inflammatory activity of a synthesized peptide, derived by treating a HaCaT cell line with TNF-α/IFN-γ (10 ng/ml) as a positive control group or TNF-α/IFN-γ in combination with a novel peptide (10 μg/ml) to identify transcription inhibitory effects of TSLP.

FIG. 3 shows a result of real-time PCR analysis conducted to identify an anti-inflammatory activity of a synthesized peptide, derived by treating a HaCaT cell line with TNF-α/IFN-γ (10 ng/ml) as a positive control group or TNF-α/IFN-γ in combination with a novel peptide (10 μg/ml) to identify transcription inhibitory effects of IL-31.

FIG. 4 shows results of identifying an effect of relieving chronic contact dermatitis after treating chronic contact dermatitis-developed mice with a novel peptide.

FIG. 5 shows a result of checking changes in the body weight after treating chronic contact dermatitis-developed mice with a novel peptide.

FIG. 6 shows results of comparatively analyzing a size of each organ after treating chronic contact dermatitis-developed mice with a novel peptide and then separating the spleen and lymph nodes, which are organs mainly involved in the immune response.

FIG. 7 shows results of analyzing, via Western blot, effects of a novel peptide (10 μg/ml) on phosphorylation of JAK2 and STAT1 which are key proteins in the JAK/STAT signaling pathway induced by TNF-α/IFN-γ (10 ng/ml) in a HaCaT cell line, in an attempt to determine a mechanism of a synthesized peptide.

### BEST MODE FOR CARRYING OUT THE INVENTION

Hereinafter, the present disclosure will be described in more detail.

A peptide preparation, a substance in which 5 to 20 amino acids which are building blocks of proteins are linked, is defined as ‘a smallest unit having a function of proteins’. The peptide preparation may regulate vital signaling and functions by selecting the smallest unit having excellent bioactivity among proteins and have distinction in terms of ‘bio-friendliness’ and ‘in vivo specificity’ as new drug candidates, thereby exhibiting strong pharmacological actions and activities even in a small amount with few side effects. In addition, compared to low-molecule-weight compounds, a success rate of new drugs in the clinical stage is more than twice. Therefore, research is actively underway to newly discover peptide preparations suitable for treatment of specific diseases and provide the same as a new drug. Accordingly, the present inventors completed the present disclosure by synthesizing a peptide in consideration of the features above and determining that the new peptide exhibits an anti-inflammatory activity in human epithelial cells.

The present disclosure may provide a pharmaceutical composition for preventing or treating inflammatory skin diseases, including a peptide having an amino acid sequence represented by SEQ ID NO: 1 as an active ingredient.

The peptide suppresses expression of one or more selected from the group consisting of CCL17, CCL22, KDM6B, GMCSF, TSLP, and IL-31.

In addition, the peptide inhibits phosphorylation of JAK2 or STAT1.

The inflammatory skin disease is one or more selected from the group consisting of, but is not limited to, contact dermatitis, allergic dermatitis, systemic lupus erythematosus, seborrheic dermatitis, psoriasis, and atopic dermatitis.

The pharmaceutical composition may reduce the inflammatory response without changes in the body weight.

In an example embodiment of the present disclosure, the pharmaceutical composition for preventing or treating an inflammatory skin disease including the peptide having the amino acid sequence represented by SEQ ID NO: 1 as an active ingredient may use any one formulation selected from the group consisting of injections, granules, powder, tablets, pills, capsules, suppositories, gels, suspensions, emulsions, drops, or liquids according to a conventional method.

In another example embodiment of the present disclosure, the pharmaceutical composition for preventing or treating an inflammatory skin disease including the peptide having the amino acid sequence represented by SEQ ID NO: 1 as an active ingredient may further include one or more appropriate additives selected from the group consisting of carriers, excipients, disintegrants, sweeteners, coating agents, swelling agents, lubricants, flavoring agents, antioxidants, buffers, bacteriostatic agents, diluents, dispersants, surfactants, binders, and lubricants that are commonly used in preparation of pharmaceutical compositions.

Specifically, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinyl pyrrolidone, water, methylhydroxy benzoate, propylhydroxybenzoate, talc, magnesium stearate, and mineral oil may be used as carriers, excipients, and diluents, and solid preparations for oral administration include tablets, pills, powder, granules, and capsules, wherein such solid preparation may be prepared by mixing, in the composition, at least one excipient such as starch, calcium carbonate, sucrose or lactose, and gelatin. In addition to simple excipients, lubricants such as magnesium stearate and talc may also be used. Liquid preparations for oral use may include suspensions, solutions, emulsions, and syrups, and various excipients such as wetting agents, sweeteners, fragrances, and preservatives may be included in addition to commonly used simple diluents such as water and liquid paraffin. Preparations for parenteral administration include sterile aqueous solutions, non-aqueous solutions, suspensions, emulsions, freeze-dried preparations, and suppositories. Propylene glycol, polyethylene glycol, vegetable oils such as olive oil, and injectable esters such as ethyl oleate may be used as non-aqueous solvents and suspensions. Witepsol, macrogol, Tween 61, cacao butter, laurin fat, and glycerogelatin may be used as a base of the suppositories.

According to an example embodiment of the present disclosure, the pharmaceutical composition is administered to a subject in a conventional manner via intravenous, intraarterial, intraperitoneal, intramuscular, intrasternal, transdermal, intranasal, inhalational, topical, rectal, oral, intraocular, or intradermal routes.

Preferred dosages of the peptide may vary depending on the subject's condition and weight, the type and severity of a disease, drug form, the route and duration of administration, and may be appropriately selected by those skilled in the art. According to an example embodiment of the present disclosure, although not limited to, the daily dose may be 0.01 to 200 mg/kg, specifically 0.1 to 200 mg/kg, and more specifically 0.1 to 100 mg/kg. Administration may be conducted once a day or in several divided doses, thereby not limiting the scope of the present disclosure.

In an example embodiment of the present disclosure, the term ‘subject’ as used herein may refer to a mammal including a human, but is not limited to the examples.

In addition, the present disclosure may provide a cosmetic composition for preventing or alleviating inflammatory skin diseases, including a peptide having an amino acid sequence represented by SEQ ID NO: 1 as an active ingredient.

The cosmetic composition may have, but is not limited to, a formulation selected from the group consisting of solutions, suspensions, emulsions, pastes, gels, creams, lotions, powders, soaps, surfactant-including cleansings, oils, powder foundations, emulsion foundations, wax foundations, and sprays.

The cosmetic composition may include, in addition to a peptide having an amino acid sequence represented by SEQ ID NO: 1 as an active ingredient, conventional adjuvants such as stabilizers, solubilizers, vitamins, pigments and fragrances, and carriers.

The cosmetic composition may be prepared in any formulation commonly manufactured in the art, including, for example, solutions, suspensions, emulsions, pastes, gels, creams, lotions, powders, oils, powder foundations, emulsion foundations, wax foundations, and sprays, but is not limited thereto. More specifically, it may be formulated as sunscreens, softening lotions, astringent lotions, nourishing lotions, nourishing creams, massage creams, essences, eye creams, packs, sprays, or powders.

If the formulation is a paste, cream or gel, animal oil, vegetable oil, wax, paraffin, starch, tracanth, cellulose derivatives, polyethylene glycol, silicone, bentonite, silica, talc, or zinc oxide may be used as a carrier component.

If the formulation is powder or a spray, lactose, talc, silica, aluminum hydroxide, calcium silicate or polyamide powder may be used as a carrier component, and in particular, in the case of the spray, chlorofluorohydrocarbon, propane/butane or a booster such as dimethyl ether may be additionally included.

If the formulation is a solution or emulsion, solvents, solubilizers or emulsifiers are used as the carrier component, for example, water, ethanol, isopropanol, ethyl carbonate, ethyl acetate, benzyl alcohol, benzyl benzoate, propylene glycol, 1,3-butylglycol oil, glycerol fatty ester, polyethylene glycol or fatty acid ester of sorbitan.

If the formulation is a suspension, liquid diluents such as water, ethanol or propylene glycol, suspensions such as ethoxy lated isostearyl alcohol, polyoxyethylene sorbitol ester, and polyoxyethylene sorbitan ester, microcrystalline cellulose, aluminum metahydroxide, bentonite, agar, or tracanth may be used as the carrier component.

### Modes For Carrying Out the Invention

Hereinafter, example embodiments will be described in detail to help the understanding of the present disclosure. However, the following example embodiments are merely illustrative of the content of the present disclosure, and the scope of the present disclosure is not limited by the following example embodiments. The example embodiments of the present disclosure are provided to more completely explain the present disclosure to those skilled in the art.

### EXAMPLE 1

### Preparation of Peptides

To synthesize a novel peptide, a liquid phase method by Merrifield using an Fmoc amino group protecting vessel (Merrifield, R B., J. Am. Chem. Soc., 85, 2149, 1963) was used to prepare the peptide.

The peptide synthesis method is a solid phase method using 9-fluorenylmethoxycarbonyl (Fmoc) as a protecting group of a Nα-amino group on amino acids, and more specifically, Rink Amide MBHA-resin was use as a starting material for the peptide with a carboxyl end in the form of-NH₂, and Fmoc-amino acid-Wang resin was used as a starting material for the peptide with a carboxyl end in the form of —OH.

The elongation of peptide chains by coupling of Fmoc-amino acids was performed by the N-hydroxy benzotriazole (HOBt)-dicyclo-hexylcar-bodiimide (DCC) method.

After linking the Fmoc-amino acids at the amino ends of each peptide, Fmoc groups were removed with a 20% piperidine/N-methylpyrrolidone (NMP) solution, and washing was performed several times with NMP and dichloromethane (DCM), followed by drying with nitrogen gas.

Therein, a solution of trifluoroacetic acid (TFA)-phenol-thioanisole-H2O-triisopropylsilane (85:5:5:2.5:2.5, vol./vol.) was added and reacted for 3 hours to remove the protecting group, the peptide was separated from the resin, and then the peptide was precipitated with diethyl ether. The crude peptide obtained by the process was purified using a purified reverse phase-HPLC column (Delta Pak, C18 300 Å, 15 μm, 19.0 mm×30 mm, Waters) with an acetonitrile gradient including 0.1% TFA.

After hydrolyzing the synthetic peptide with 6 N-HCl at 110° C. for 24 hours, the obtained residue was concentrated under reduced pressure and dissolved in 0.02 N-HCl, and the amino acid composition was measured by an amino acid analyzer (Hitachi 8500 A).

In addition, the molecular weight was calculated based on the sequence of the synthesized peptides, and the exact molecular weight was measured using the matrix assisted laser desorption ionization (MALDI) mass spectrometer.

As a result, it was found that the measured molecular weight and the calculated molecular weight matched, and thus the synthesis of KTFR peptide (SEQ ID NO: 1) with the correct amino acid sequence was determined.

### EXAMPLE 2

### Identification of an Anti-Inflammatory Activity of Peptides

### 1. Identification of Transcription Inhibitory Effects of CCL17, CCL22, KDM6B. and GMCSF

To determine whether the previously synthesized novel peptide may exhibit an anti-inflammatory activity on dermatitis, the transcription inhibitory effects on CCL17, CCL22, KDM6B, and GMCSF were identified.

First, a HaCaT cell line, which is a human epidermal cell, was cultured at 37° C. in the presence of 5% CO₂ using a DMEM medium including 1% penicillin-streptomycin and 10% FBS.

Using the DMEM medium with 10% FBS added to a 6-well cell culture plate, HaCaT was dispensed by 3×10⁵/well. Cells were cultured for one day and treated with TNF-α/IFN-γ (10 ng/ml, positive control) alone or simultaneously with TNF-α/IFN-γ and KTFR peptide (10 μg/ml) using a serum-free medium, followed by culture for 24 hours.

After 24 hours, the media were removed from each well, washing was performed with D-PBS, RNA was isolated using the usual method using RNA-prep (Bio-Zol, MA500, BIOSESANG) solution, and cDNA was synthesized using 5 μg of isolated RNA.

PCR was performed using the synthesized cDNA and primers for the target gene as shown in Table 1, and the results were observed after electrophoresis using a 1.2% agarose gel.

TABLE 1

| Cate- | | |
|---|---|---|
| gory | Forward Primer | Reverse Primer |
| CCL17 | actgtctcccgggactacct | tccctcactgtggctcttct |
| CCL22 | tgccctgggtgaagatgatt | agaggatgggttagaggggt |
| KDM6B | cccttcacatggcagtag | agagttgcagcctctcct |
| GMCSF | agagcctgctgctcttg | cagtcaaaggggatgaca |

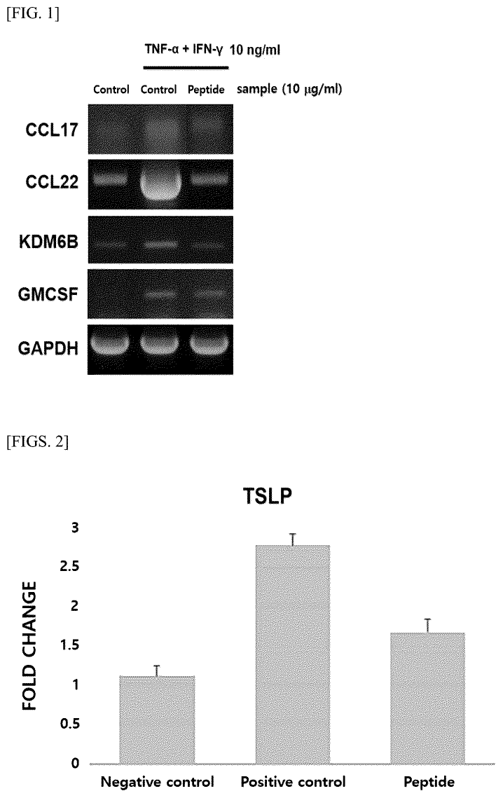

As a result, transcription inhibition for CCL17, CCL22, KDM6B, and GMCSF was observed in the experimental group treated with TNF-α/IFN-γ and the novel peptide together as shown in FIG. 1.

### 2. Identification of Transcription Inhibitory Effects of TSLP and IL-31

In the same method as the previous experiment, the HaCaT cell line, which is a human epidermal cell, was cultured at 37° C. in the presence of 5% CO2 using a DMEM medium including 1% penicillin-streptomycin and 10% FBS.

Using a DMEM medium with 10% FBS added to a 6-well cell culture plate, HaCaT was dispensed by 3×10⁵/well. Cells were cultured for one day and treated with TNF-α/IFN-γ (10 ng/ml, positive control) alone or simultaneously with TNF-α/IFN-γ and KTFR peptide (10 μg/ml) using a serum-free medium, followed by culture for 24 hours.

After 24 hours, the media were removed from each well, washing was performed with D-PBS, RNA was isolated by applying a usual method using RNA-prep (Bio-Zol, MA500,BIOSESANG) solution, and cDNA was synthesized using 5 μg of isolated RNA. With the synthesized cDNA, real-time PCR was performed using the primer of the target gene as shown in Table 2 and SYBR green, and the Ct value was determined.

TABLE 2

| Cate- | | |
|---|---|---|
| gory | Forward Primer | Reverse Primer |
| TSLP | ggggctaaaccatgacagaa | gtttggctgaaggcttgttc |
| IL31 | cgacgtctgtgctctttctg | agcatcttcgagagggactg |

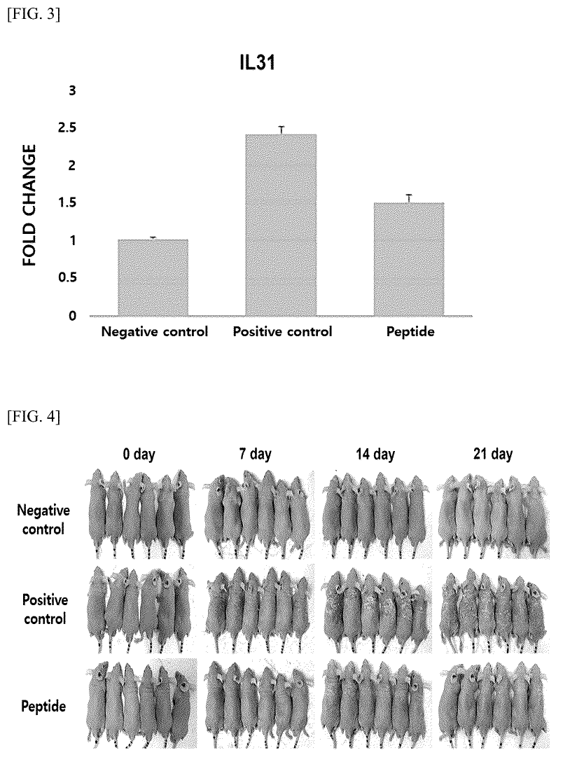

As a result, TSLP and IL-31 reduction was found to be highly effective in cell groups treated with the novel peptide, as shown in FIG. 2 and FIG. 3.

### EXAMPLE 3

### Animal Experiment

The 6-week-old male BALB/c mouse used in the animal experiment is a species of ‘SKH-1 hairless’ and barely has body hair to ensure convenience in intuitively observing the skin condition, of which was divided, for experiment, into 3 groups out of 18 individuals in total (6 individuals for a negative control group/6 individuals for a positive control group/6 individuals for a novel peptide treated group).

After an adaptation period of about 2 weeks, 100 μL of 2,4-dinitrochlorobenzene (DNCB) prepared at a concentration of 1% in solution of acetone: olive oil (3:1) was applied onto the dorsal skin of mice at a certain time every day. After 1 week, if the incidence of contact dermatitis was observed, the DNCB was lowered to 0.5% and applied by 100 μL every 2 days, and the novel peptide was prepared in normal saline at a concentration of 1 mg/ml and applied by 100 μg daily for 3 weeks, while skin condition was observed every week during the experiment.

As a result, as shown in FIG. 4, severe skin inflammation was induced in all groups except the negative control group after the adaptation period for the experimental animals, but it was found that symptoms of skin inflammation in the novel peptide treated group were significantly relieved from a short treatment period of about a week and that symptoms of skin inflammation were alleviated to almost the same extent as normal skin after a total of 3 weeks of treatment.

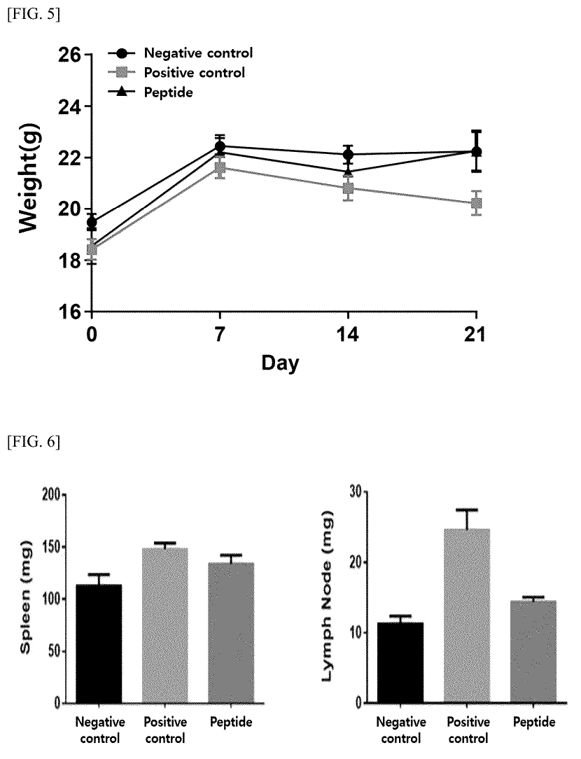

In addition, as a result of checking changes in the body weight in the negative control group, positive control group, and novel peptide treated group, no significant changes in the body weight were found in all groups, as shown in FIG. 5.

From the above results, it was found that no stress was induced by the novel peptide.

On the other hand, in order to determine the effect of novel peptide treatment on the spleen and lymph nodes, organs that are primarily involved in the immune response, the spleen and lymph nodes were separated by sacrificing animal models with contact dermatitis to comparatively analyze the size of each organ.

As a result, when the size of each organ was compared with the negative control group as shown in FIG. 6, it was found that the size increased considerably in the positive control group as immune cells were deposited in the spleen and lymph nodes, while in the group treated with the novel peptide, the size of the spleen and lymph nodes decreased remarkably to a degree similar to the negative control group, compared to the positive control group.

From the above results, it was found that the novel peptide has an excellent inhibitory effect on the overall inflammatory response in addition to contact dermatitis.

### EXAMPLE 4

### Identification of Inhibitory Effects of Peptides on Phosphorylation of Key Proteins Involved in the Signaling Pathway Induced by TNF-α/IFN-γ Treatment

In order to identify the mechanism of the novel peptide in the inflammatory response, the phosphorylation of key proteins involved in the signaling pathway, which is a regulating factor in the expression of inflammation-related cytokines and chemokines, was identified by Western blot. Using the DMEM medium with 10% FBS added to a 6-well cell culture plate, HaCaT was dispensed by 3×10⁵/well. Cells were cultured for one day and treated with TNF-α/IFN-γ (10 ng/ml, positive control) alone or simultaneously with TNF-α/IFN-γ and KTFR peptide (10 μg/ml) using a serum-free medium, followed by culture for 24 hours.

After 24 hours, the media were removed from each well, washing was performed with D-PBS, cells were lysed with 200 μl of RIPA buffer on ice, and the lysate was centrifuged (15,000 rpm, 4° C., 20 min) to isolate proteins. Protein concentrations were determined by the Bradford method, and the same amount of protein (40 μg) was isolated by 10% SDS-PAGE. The isolated proteins were transferred to a nitrocellulose membrane, and Western blot was performed using antibodies that specifically recognize the phosphorylated key proteins and the total key proteins.

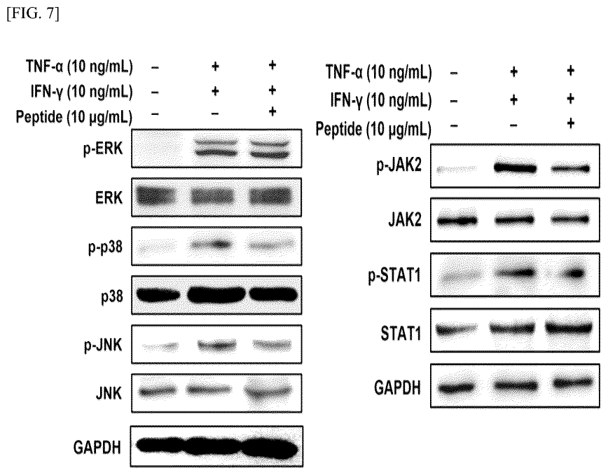

As a result, as shown in FIG. 7, the phosphorylation of the key proteins in the MAPK/ERK signaling pathway among the signaling pathways induced by TNF-α/IFN-γ treatment did not change during the treatment of the novel peptide, but the phosphorylation of JAK2 and STAT1, the key proteins in the JAK/STAT signaling pathway, was significantly reduced when the novel peptide was treated.

From the above results, it was determined that the effect of the novel peptide on the inhibition of production of related inflammatory mediators and cytokines was mediated through inhibition of the JAK/STAT signaling pathway.

Having described specific parts of the present disclosure in detail above, it would be clear to those skilled in the art that these specific descriptions are merely preferred example embodiments and the scope of the present disclosure is not limited thereby. Accordingly, the substantial scope of the present disclosure will be defined by the appended claims and equivalents thereof.

1. A method of preventing or treating inflammatory skin diseases, comprising:
administering a pharmaceutical composition comprising a peptide having an amino acid sequence represented by SEQ ID NO: 1 as an active ingredient to a subject.

2. The method of claim 1, wherein the peptide suppresses expression of one or more selected from the group consisting of CCL17, CCL22, KDM6B, GMCSF, TSLP, and IL-31.

3. The method of claim 1, wherein the peptide inhibits phosphorylation of JAK2 or STAT1.

4. The method of claim 1, wherein the inflammatory skin disease is one or more selected from the group consisting of contact dermatitis, allergic dermatitis, systemic lupus erythematosus, seborrheic dermatitis, psoriasis, and atopic dermatitis.

5. A method of preventing or alleviating inflammatory skin diseases, comprising: administering a cosmetic composition comprising a peptide having an amino acid sequence represented by SEQ ID NO: 1 as an active ingredient to a subject.

6. The method of claim 5, wherein the cosmetic composition has a formulation selected from the group consisting of solutions, suspensions, emulsions, pastes, gels, creams, lotions, powders, soaps, surfactant-including cleansings, oils, powder foundations, emulsion foundations, wax foundations, and sprays.


SEQUENCE LISTING (13 sequences)

SEQ ID NO: 1 - protein, 4 aa, Artificial Sequence
  Synthetic peptide
KTFR

SEQ ID NO: 2 - DNA, 20 bp, Artificial Sequence
  Forward Primer (CCL17)
actgtctcccgggactacct

SEQ ID NO: 3 - DNA, 20 bp, Artificial Sequence
  Reverse Primer (CCL17)
tccctcactgtggctcttct

SEQ ID NO: 4 - DNA, 20 bp, Artificial Sequence
  Forward Primer (CCL22)
tgccctgggtgaagatgatt

SEQ ID NO: 5 - DNA, 20 bp, Artificial Sequence
  Reverse Primer (CCL22)
agaggatgggttagaggggt

SEQ ID NO: 6 - DNA, 18 bp, Artificial Sequence
  Forward Primer (KDM6B)
cccttcacatggcagtag

SEQ ID NO: 7 - DNA, 18 bp, Artificial Sequence
  Reverse Primer (KDM6B)
agagttgcagcctctcct

SEQ ID NO: 8 - DNA, 17 bp, Artificial Sequence
  Forward Primer (GMCSF)
agagcctgctgctcttg

SEQ ID NO: 9 - DNA, 18 bp, Artificial Sequence
  Reverse Primer (GMCSF)
cagtcaaaggggatgaca

SEQ ID NO: 10 - DNA, 20 bp, Artificial Sequence
  Forward Primer (TSLP)
ggggctaaaccatgacagaa

SEQ ID NO: 11 - DNA, 20 bp, Artificial Sequence
  Reverse Primer (TSLP)
gtttggctgaaggcttgttc

SEQ ID NO: 12 - DNA, 20 bp, Artificial Sequence
  Forward Primer (IL31)
cgacgtctgtgctctttctg

SEQ ID NO: 13 - DNA, 20 bp, Artificial Sequence
  Reverse Primer (IL31)
agcatcttcgagagggactg